(19) United States
(12) Patent Application Publication
(10) Pub. No.: US 2026/0083906 A1
(43) Pub. Date: Mar. 26, 2026

(54) Mechanisms for Thyroid Hormone Delivery

(71) Applicant: Brian Haney, Reno, NV (US)

(72) Inventor: Brian Haney, Reno, NV (US)

(21) Appl. No.: 18/892,392

(22) Filed: Sep. 21, 2024

(51) Int. Cl. A61M 5/142; A61K 31/195; A61M 5/172

(52) U.S. Cl. CPC A61M 5/14276; A61K 31/195; A61M 5/172

(57) ABSTRACT

The present disclosure provides methods and a device for thyroxine delivery. This device is designed for subcutaneous implantation to ensure continuous delivery of thyroxine. It features a reservoir regulated by an embedded microprocessor with advanced signal software. The software triggers a precision pump to release a specific dose of thyroxine at regular intervals. This system enhances patient compliance and maintains optimal metabolic control.

### CROSS REFERENCE TO RELATED APPLICATIONS

Not applicable.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### INCORPORATION BY REFERENCE OF MATERIAL SUBMITTED

Not applicable.

### STATEMENT REGARDING PRIOR DISCLOSURES BY THE INVENTOR

Not applicable.

### BACKGROUND TO THE INVENTION

### Field of the Invention

The field of the invention rests at the intersection of two broader fields, biotechnology and endocrinology. Endocrinology is a medical branch concerned with endocrine glands and hormones, including the thyroid and thyroid hormones. Biotechnology is any new and better machine or mechanism for improving the quality of human life or treating disease. Specifically, the field of the invention is drug delivery to patients with hypothyroidism, the most common thyroid disease in the United States.

### Background Art

Hypothyroidism is an endocrine disease affecting an estimated 9,900,000 people in the United States, 37,070,000 in Europe, and 104,000,000 people globally. Conventional methods for curing the disease fail for various reasons leading to ongoing patient symptoms including weight gain, depression, and anxiety. Further, medical reports suggest a common trend is that more than half of patients with hypothyroidism are undiagnosed and untreated. Moreover, there are several hypothyroidism types. For example, Congenital Hypothyroidism (CH) is defined as thyroid hormone deficiency present at birth. Another example is Hashimoto's Disease, a hypothyroid condition in which the immune system attacks the human thyroid gland preventing adequate Thyroxine (T₄) production.

The thyroid is a bilobal gland located at the base of the neck in front of the windpipe. The thyroid's functionality, essentially synthesizing thyroid hormone, is meticulously modeled through a feedback loop. The pituitary gland and thyroid communicate instructions for controlling hormone production and stabilization. The system works on an iterative loop where cells in the pituitary gland determine the body's normal hormonal range, known as the set point. In other words, the thyroid produces hormones, which are secreted into the blood and then carried to every tissue in the body. As such, the Thyroid produces a hormonal variety governing the human body's metabolism.

Thyroid hormone synthesis is a three-step process. First, the hypothalamus produces Thyroid Releasing Hormone (TRS), stimulating the pituitary gland to release Thyroid Stimulating Hormone (TSH), Thyrotropin, which in turn activates the thyroid gland. Second, the thyroid gland excretes Thyroxine (T₄) to the bloodstream. Third, T₄ converts to Triiodothyronine (T₃) through deiodination in peripheral tissues. This synthesis is critical for metabolic control in the human body.

Thyroxine (T₄) converts to the active Triiodothyronine (T₃) within cells and peripheral tissues by deiodinases. As such, in contrast to Thyroxine (T₄), the Triiodothyronine (T₃) molecule contains three iodine atoms. Triiodothyronine (T₃) is the physiologically active thyroid hormone. It controls myocardium properties, heart rate, and vascular function. In fact, Triiodothyronine (T₃) affects almost every process in the body. Some suggest the thyroid gland produces T₃ directly. Although, thyroid disease is typically not treated with Triiodothyronine (T₃) supplementation. However, speculate a Thyroxine (T₄) and Triiodothyronine (T₃) combination might be better. Molecular structures are important because clinical effects resulting from thyroid hormone imbalance are observable at the cellular level.

As such, Thyroid hormone maintenance is extremely critical for adult metabolic activity, and thyroid hormone abnormalities in adolescence can have catastrophic consequences. Thyroid hormone imbalance can have profound effects on the central nervous system. Interestingly, Thyroid hormone receptors are located throughout the brain, highlighting their importance in central nervous system's development and function. Indeed, Triiodothyronine (T₃) and Thyroxine (T₄) also provide feedback to the brain and anterior pituitary gland to regulate thyroid hormone.

In adults, Hypothyroid symptoms include chronic fatigue, depression, impaired memory, and weight gain. In adolescents, Hypothyroid symptoms include poor growth trajectories, limited mental development, and delayed puberty. Additionally, hypothyroidism may cause problems such as sleep apnea, hypothermia, hypoventilation, neuropsychiatric syndromes, peripheral neuropathy, seizure, cerebellar ataxia, and coma.

Thyroid hormone replacement therapy is a chronic and lifetime endeavor for treatment. Thyroid hormone dosage must be established for each patient Individually. Usually, the initial dose is small, with amounts increasing gradually until clinical evaluation and laboratory tests indicate optimal response. The dose required to maintain this response is then continued. It is vital to patients physical and mental health that thyroid hormone treatment have the correct dosage. Under-treatment with thyroid hormone can have deleterious and disastrous consequences including neurochemical imbalances, depression, and fatigue.

Prior to the disclosed invention, the art reflected the idea that hypothyroidism could not be cured. Rather, since the year 1970s, the conventional wisdom has been that hypothyroidism is only treatable by replacing thyroid hormone deficiencies with orally administered levothyroxine (LT₄). And orally administered levothyroxine (LT₄) is still the standard treatment for hypothyroid patients despite its primitive conception five decades ago.

One major issue is the absorption variability of LT4. The bioavailability of orally administered LT4 can be influenced by various factors, including the timing of the dose, the presence of food, and the use of certain medications. For instance, taking LT4 with food, especially those high in fiber, calcium, or iron, can significantly reduce its absorption, leading to suboptimal therapeutic effects. This necessitates strict adherence to guidelines, such as taking LT4 on an empty stomach and waiting at least 30 to 60 minutes before eating, which can be challenging for patients to consistently follow.

Another problem is the interaction between LT4 and other medications or supplements. Commonly used substances like calcium carbonate, iron supplements, proton pump inhibitors, and certain cholesterol-lowering drugs can interfere with LT4 absorption. These interactions require careful timing and separation of LT4 intake from other medications, adding complexity to the treatment regimen and increasing the risk of non-adherence. Additionally, some conditions, such as gastrointestinal disorders like celiac disease or lactose intolerance, can impair LT4 absorption, necessitating higher doses or alternative administration methods.

Moreover, individual variability in LT4 metabolism and clearance can affect treatment outcomes. Factors such as age, body weight, hormonal changes, and genetic differences can influence how LT4 is processed in the body. This variability means that some patients may require frequent dose adjustments and regular monitoring of thyroid-stimulating hormone (TSH) levels to achieve and maintain homeostasis. Such ongoing adjustments can be burdensome for both patients and healthcare providers, potentially leading to frustration and decreased quality of life for those affected.

Direct drug delivery devices represent a transformative advancement in the field of medicine, designed to deliver therapeutic agents precisely to targeted areas within the body. These devices aim to enhance the efficacy and safety of treatments by optimizing the concentration of drugs at specific sites, thereby minimizing systemic exposure and reducing side effects. The evolution of these devices has been driven by the need to address limitations associated with traditional drug delivery methods, such as oral ingestion or intravenous injection, which often result in suboptimal drug distribution and a higher likelihood of adverse reactions.

The concept of direct drug delivery can be traced back to early attempts to localize treatment for certain conditions, such as the use of topical creams for skin diseases or eye drops for ocular conditions. However, significant technological advancements over the past few decades have revolutionized this field. Innovations such as micro-and nanotechnology, smart polymers, and bioengineered materials have enabled the development of sophisticated delivery systems that can navigate the complex biological environment of the human body. These devices can be designed to respond to specific stimuli, such as pH changes, temperature variations, or the presence of certain enzymes, ensuring that the drug is released only at the desired location and at the right time.

Examples of direct drug delivery devices include implantable pumps, drug-eluting stents, and targeted nanoparticles. Implantable pumps can be used to administer pain medication or insulin continuously and directly to a specific site, ensuring consistent therapeutic levels. Drug-eluting stents, commonly used in cardiovascular treatments, release drugs slowly to prevent artery blockage post-surgery. Targeted nanoparticles can deliver chemotherapy agents directly to cancer cells, sparing healthy tissue and reducing side effects. These advancements highlight the potential of direct drug delivery devices to improve patient outcomes significantly, providing more effective and personalized therapeutic options for a wide range of medical conditions.

There are three major problems with daily oral levothyroxine for treating hypothyroidism: (1) fluctuation in ingestion times and surrounding dietary conditions causes metabolic imbalance, (2) fluctuation in patient needs causes metabolic imbalance, and (3) fluctuation in brand name may cause metabolic imbalance. Thurs, there exists a need for new medical methods for treating patients with hypothyroidism. The disclosed invention solves these three problems, providing mechanisms for delivering thyroid hormones with automatic and corrective release to help patients maintain metabolic homeostasis.

### SUMMARY OF THE INVENTION

The invention is mechanisms for thyroid hormone delivery. The mechanisms include methods and an apparatus or direct drug delivery. The methods include distributing thyroid hormone on a timed basis to a patient. The apparatus is an implantable device with a thyroid hormone store and pump to deliver the hormone to the patient. The mechanisms work together to optimize patient metabolic homeostasis and treat hypothyroidism.

In certain embodiments, the present disclosure is an apparatus for thyroid hormone delivery. In such embodiments, the method may comprise an implantable device with a cylindrical shape. The implantable device may further comprise a microprocessor with embedded instructions for commanding a thyroid hormone pump. The thyroid hormone pump may receive a signal from the microprocessor to pump thyroid hormone supply from a thyroid hormone supply. The thyroid hormone supply may then be pumped into the human body at interval times to optimize human metabolic and thyroid hormone homeostasis.

In certain embodiments, the present disclosure is a method for thyroxine delivery. The method may comprise signaling the delivery of the thyroxine on a timed basis. Next, the method may include sending a command to a thyroid hormone pump. Then, the method may comprise pumping the thyroxine by a thyroid hormone pump. In certain embodiments, the final step may be delivering thyroxine to the human body and maintaining metabolic homeostasis in the human body.

In certain embodiments, the disclosure is a process and device for treating hypothyroidism using artificial intelligence to measure blood and command hormonal secretion through a valve delivery mechanism. First, blood sensors measure hormonal concentration in the blood, storing measurements in memory for further processing the measurement. The sensors measure blood to detect and commit hormone levels to memory, including: Thyroxine (T₄), Triiodothyronine (T₃), Free Thyroxine (FT₄), and other thyroid hormones. Second, the measurements are processed and compared to target hormonal levels, and subsequently produce an automatic command for a valve control and drug delivery mechanism. Third, the valve control subsystem motivates thyroid hormone delivery from a supply of reserved hormone in a module to the patient's bloodstream optimizing hormonal homeostasis.

### BRIEF DESCRIPTION OF THE DRAWINGS

FIG. 1 is an illustration of an apparatus for thyroid hormone delivery.

FIG. 2 is an illustration for a method of thyroid hormone delivery.

FIG. 3 is an illustration for a method of thyroid hormone delivery.

### DETAILED DESCRIPTION OF THE INVENTION

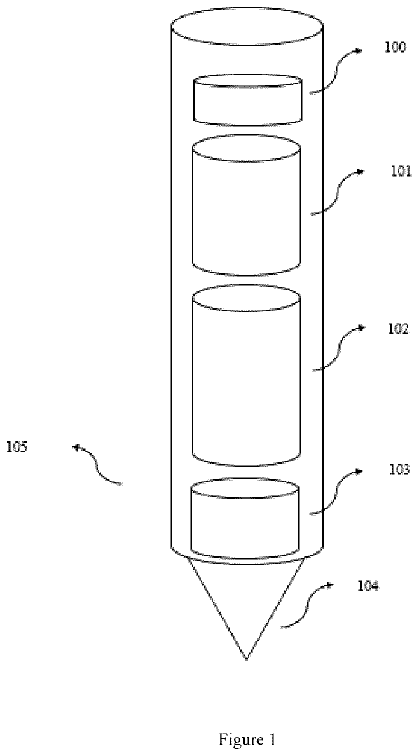

FIG. 1 is an illustration of an apparatus for thyroid hormone delivery. In certain embodiments, a microprocessor 100 sends a signal to a pump 101. The pump creates pressure on a thyroid hormone supply 102, which moves thyroid hormone to a secretion chamber 103. Finally, the thyroid hormone in the secretion chamber moves to a human body via an injection device 104. The complete device operates a synthetic thyroid apparatus 105, replacing or supplementing the functionality of the human thyroid.

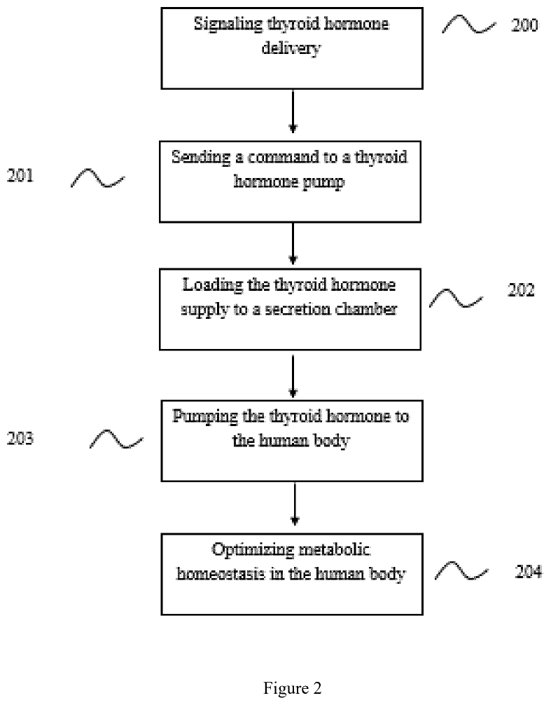

FIG. 2 is an illustration for a method of thyroid hormone delivery. In certain embodiments, the disclosure is a process for thyroid hormone delivery. The process begins with the signaling of thyroid hormone delivery 200. Next, the process proceeds with sending a command of thyroid hormone pump 201. Then, the thyroid hormone supply is loaded to a secretion chamber 202. Next, the thyroid is pumped to the human body 203 to optimize metabolic homeostasis 204.

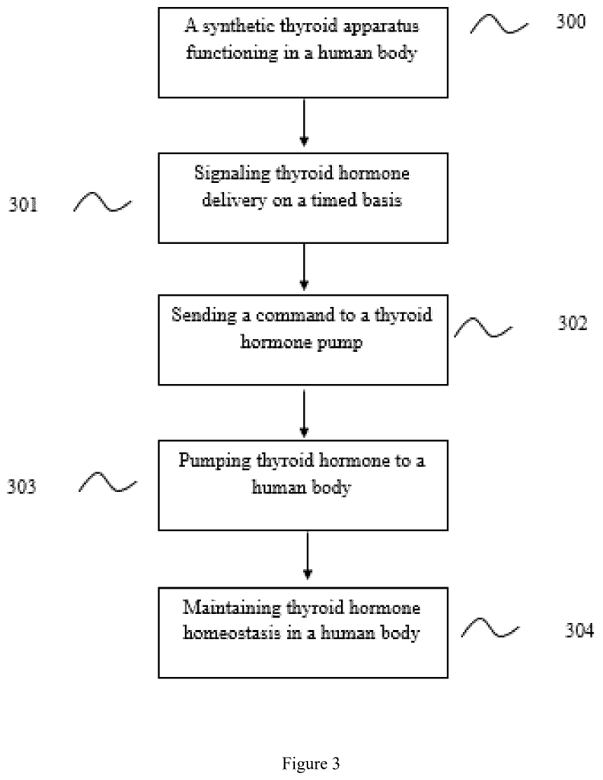

FIG. 3 is an illustration for a method of thyroid hormone delivery. In certain embodiments, the disclosure is a process for thyroid hormone delivery. In such embodiments, the process begins with signaling thyroid hormone delivery on a timed basis 300. The process proceeds with sending a command to a thyroid hormone pump 301 and loading a thyroid hormone supply to a secretion chamber 302. Then, the thyroid hormone is pumped to the human body 303 to maintain thyroid hormone homeostasis in the human body 304.

In certain embodiments, the present disclosure is a method for thyroxine delivery. The method may comprise signaling the delivery of the thyroxine on a timed basis. Next, the method may include sending a command to a thyroid hormone pump 101. Then, the method may comprise pumping the thyroxine by a thyroid hormone pump 203. In certain embodiments, the final step may be delivering thyroxine to the human body and maintaining metabolic homeostasis in the human body 304.

In certain embodiments, the present disclosure is a method for thyroid hormone delivery. This method involves signaling the delivery of thyroid hormone on a timed basis 200. It includes sending a command to a thyroid hormone pump, which then pumps the thyroid hormone 303. The thyroid hormone is delivered to the human body through this pump, ultimately maintaining thyroid hormone homeostasis within the body.

In certain embodiments, the present disclosure is a method for delivering thyroid hormones via a synthetic thyroid apparatus 105. This method begins by signaling the delivery of thyroid hormone at specified intervals, ensuring that the timing of hormone administration is carefully controlled. A command is then sent to a thyroid hormone pump, which is responsible for dispensing the hormone 201. The pump activates and releases the thyroid hormone into the human body according to the pre-determined schedule. This precise delivery system ensures that thyroid hormone levels are regulated effectively, thereby optimizing thyroid hormone homeostasis within the body 204.

In certain embodiments, the present disclosure is a method for thyroid hormone delivery comprising several steps. The first step is signaling the delivery of the thyroid hormone on a timed basis 200. The second step is sending a command to a thyroid hormone pump 101. The third step is pumping the thyroid hormone by a thyroid hormone pump 203. The final step is delivering thyroid hormones to the human body and maintaining thyroid hormone homeostasis in the human body 304.

In certain embodiments, the present disclosure is an apparatus for thyroid hormone delivery. The apparatus may be an implantable device with a cylindrical shape. The implantable device may include a microprocessor 100 with embedded instructions for commanding a thyroid hormone pump. The apparatus may also include a thyroid hormone pump, receiving a signal from the microprocessor to pump thyroid hormone supply 102. The apparatus may also include a thyroid hormone supply, the thyroid hormone supply being pumped into the human body by the thyroid hormone pump 203.

In certain embodiments, the present disclosure is an apparatus for thyroid hormone delivery. In such embodiments, the method may comprise an implantable device with a cylindrical shape. The implantable device may further comprise a microprocessor 100 with embedded instructions for commanding a thyroid hormone pump 101. The thyroid hormone pump may receive a signal from the microprocessor to pump thyroid hormone supply 301 from a thyroid hormone supply 102. The thyroid hormone supply may then pumped into the human body at interval times to optimize human metabolic and thyroid hormone homeostasis 204.

In certain embodiments, the present disclosure is an apparatus for thyroid hormone delivery. This apparatus includes an implantable device with a cylindrical shape, designed for efficient integration into the human body. The device is equipped with a microprocessor 100 that contains embedded instructions to control a thyroid hormone pump 101. Upon receiving a signal from the microprocessor, the thyroid hormone pump activates and dispenses the thyroid hormone supply 102. The thyroid hormone supply is then pumped into the human body 203, ensuring consistent and regulated delivery of the hormone as needed.

In certain embodiments, the present disclosure is an apparatus designed for the delivery of thyroid hormones. This apparatus features an implantable device with a cylindrical shape, engineered to be discreetly inserted into the human body. The device houses a microprocessor 100 equipped with embedded instructions that precisely control the operation of a thyroid hormone pump. When the microprocessor sends a command, the thyroid hormone pump activates, drawing from a stored thyroid hormone supply. This supply is then meticulously pumped into the human body, ensuring that the hormone levels are maintained at optimal levels 204. The entire system is designed to provide consistent and regulated hormone delivery, contributing to effective thyroid hormone management and homeostasis.

In certain embodiments, the present disclosure is a method for thyroid hormone delivery. The method may comprise signaling the delivery of the thyroid hormone on a timed basis, wherein the time basis is on a twelve-hour interval. The instruction may be to send a command to a thyroid hormone pump 202, where the thyroid hormone supply is loaded by the pump to a secretion chamber 103 and pumped thyroid hormones from a secretion chamber to the human body by the thyroid hormone pump. The purpose is to optimize metabolic homeostasis in the human body 204.

In certain embodiments, the present disclosure is a method for thyroid hormone delivery. The method may comprise signaling the delivery of the thyroid hormone on a timed basis. The time basis may be a twelve-hour interval. The process may proceed by sending a command to a thyroid hormone pump 302, loading the thyroid hormone supply by the pump to a secretion chamber, and pumping thyroid hormones from a secretion chamber to the human body by the thyroid hormone pump. The result of the process is optimizing metabolic homeostasis in the human body 204.

In certain embodiments, the present disclosure is a method for thyroid hormone delivery. In such embodiments, the method comprises signaling the delivery of thyroid hormone on a timed basis 200. The time basis may be a twenty-four-hour interval. The process may proceed by sending a command to a thyroid hormone pump 302, loading the thyroid hormone supply by the pump to a secretion chamber, and pumping thyroid hormones from a secretion chamber to the human body by the thyroid hormone pump. The result of the process is optimizing metabolic homeostasis in the human body 204.

In certain embodiments, the present disclosure is a method for thyroid hormone delivery. In such embodiments, the method comprises signaling the delivery of thyroid hormone on a timed basis 200. The time basis may be an eight-four-hour interval. The process may proceed by sending a command to a thyroid hormone pump 302. The thyroid hormone may then be loaded by the pump to a secretion chamber 103. The pump may then apply pressure to the thyroid hormone in the secretion chamber, pumping thyroid hormone from a secretion chamber to the human body by the thyroid hormone pump. The result of the process is optimizing metabolic homeostasis in the human body 204.

In certain embodiments, the disclosure is a device for treating hypothyroidism with thyroxine drug administration. The device comprises a sensor measuring thyroxine in the human body using a thyroid hormone sensor, sending the measurement data to a microchip computer processor. The microchip computer processor further comprises an embedded computer program comprising instructions, the instructions provide for calculating optimum levels of thyroxine in the human body, comparing the optimum calculated levels of thyroxine to the measured levels of thyroxine, predicting needed delivery dosage between a range of four micrograms and three-hundred-and-one micrograms, and sending the predicted needed delivery dosage to a second computer program. The second computer program commands drug administration to the human body from a stored thyroid hormone supply, administering thyroxine once every twelve hours, according to the dosage defined by the computer program comprising a set of logical instructions defined by medical experts, helping the patient maintain metabolic homeostasis by measuring and administering thyroid hormone in the human body.

In certain embodiments, the disclosure is a method for treating hypothyroidism with thyroxine drug administration. The method comprises measuring thyroxine in the human body using a thyroid hormone sensor and sending the measurement data to a microchip computer processor. The microchip computer processor further comprises an embedded computer program comprising instructions, defined by medical experts. The computer program calculates optimum levels of thyroxine in the human body, compares the optimum calculated levels of thyroxine to the measured levels of thyroxine, predicts needed delivery dosage between a range of four micrograms and three-hundred-and-one micrograms, and sends the predicted needed delivery dosage to a second computer program. The second computer program commands drug administration to the human body from a stored thyroid hormone supply, administering thyroxine once every twelve hours, according to the dosage defined by the computer program, helping the patient maintain metabolic homeostasis by measuring and administering thyroid hormone in the human body.

In certain embodiments, the present disclosure is an apparatus for thyroid hormone delivery. This apparatus operates by signaling the delivery of thyroid hormone on a timed basis, with the intervals set to every twelve hours. The system sends a command to a thyroid hormone pump, which then loads the thyroid hormone supply into a secretion chamber. From this chamber, the thyroid hormones are pumped into the human body by the pump. This controlled delivery mechanism ensures the optimization of metabolic homeostasis within the body, maintaining balanced thyroid hormone levels for effective physiological regulation.

In certain embodiments, the present disclosure is an apparatus for thyroid hormone delivery. This apparatus is designed to deliver thyroid hormones through a precise and regulated process. It begins by signaling the delivery of thyroid hormone at set intervals, specifically every twelve hours, ensuring consistent administration. The apparatus sends a command to a thyroid hormone pump, which is integral to the system. Upon receiving this command, the pump loads the thyroid hormone supply into a dedicated secretion chamber. Once the hormone supply is in the chamber, the pump then dispenses the thyroid hormones into the human body. This methodical process is carefully engineered to optimize metabolic homeostasis, ensuring that thyroid hormone levels are maintained within a healthy range and contributing to overall physiological balance.

In certain embodiments, the present disclosure describes a microprocessor-controlled titanium tube designed for the precise delivery of thyroxine into the body. This small, implantable device integrates advanced microelectronics and biocompatible materials to provide an efficient and reliable solution for hormone replacement therapy. At its core, the device features a compact microprocessor programmed to regulate thyroxine release with high precision, utilizing sensors to monitor physiological parameters such as hormone levels and patient activity. This enables real-time feedback and adaptive control, allowing for personalized dosing schedules based on the patient's needs. The thyroxine is encapsulated in a miniature titanium tube, chosen for its excellent biocompatibility, corrosion resistance, and durability, ensuring the hormone remains stable and protected. The tube houses a precision-controlled pump, activated by the microprocessor, which delivers the hormone accurately and consistently into the bloodstream. This pumping mechanism operates silently and efficiently, minimizing patient discomfort while maintaining a steady hormone dose. A long-lasting battery powers the device, designed to minimize power consumption and extend operational life, making it a robust solution for long-term hormone replacement therapy.

In certain embodiments, the present disclosure addresses the efficient absorption of thyroxine once delivered into the body. The thyroxine released from the implant is designed to be bioavailable and readily absorbable by the surrounding tissues. Upon release, thyroxine diffuses through the interstitial fluid and reaches nearby capillaries, entering the bloodstream. The highly vascularized nature of the implantation site ensures rapid uptake of the hormone into the circulatory system.

In such embodiments, once in the bloodstream, thyroxine binds to plasma proteins, primarily thyroxine-binding globulin, which transports it to target tissues throughout the body. The hormone is then absorbed by cells, where it undergoes conversion to its active form, triiodothyronine (T3), within the cells' cytoplasm. This active form interacts with nuclear receptors, initiating the transcription of specific genes that regulate metabolism, growth, and development.

In such embodiments, the controlled release mechanism of the implant ensures a steady and consistent level of thyroxine in the bloodstream, preventing the peaks and troughs associated with conventional oral dosing. This steady state of hormone levels facilitates optimal absorption and utilization by the body's tissues, enhancing the therapeutic efficacy of the treatment and improving overall metabolic stability. By maintaining precise control over thyroxine release and absorption, the implant supports a more balanced and effective management of thyroid hormone levels, ultimately promoting better health outcomes for patients with hypothyroidism.

The pump mechanism in the microprocessor-controlled titanium tube implant offers a precise solution to the burst release problem commonly seen in polymer implants. Unlike traditional polymer matrices that rely solely on passive degradation to release the drug, the pump actively controls the release of thyroxine, allowing for a highly regulated and consistent delivery profile.

Upon implantation, the microprocessor monitors the patient's physiological conditions and determines the optimal release schedule. The pump then dispenses the thyroxine in carefully measured doses, ensuring that the hormone is released steadily over time rather than in an initial burst. This active control mechanism effectively mitigates the risk of an initial surge in hormone levels, which can occur with polymer-only systems as they begin to degrade.

Moreover, the pump's precision in dosage administration allows for fine-tuning of the release rates, which can be adjusted based on real-time feedback from the patient's hormone levels and metabolic needs. This adaptability not only prevents the burst release but also ensures that the patient maintains a stable and therapeutic level of thyroxine, thereby enhancing the efficacy and safety of the treatment. The use of a pump thus represents a significant advancement over traditional polymer implants, providing a reliable and controlled method for hormone delivery.

To make the implant adjustable for dosages, the device can be integrated with a carbon-coded material that interacts with the microprocessor to finely control the release of thyroxine. This advanced material, characterized by its excellent conductivity and responsiveness to electrical signals, allows for precise modulation of the pump's activity based on real-time data. Upon implantation, the microprocessor, embedded within the titanium tube, continuously monitors the patient's physiological parameters through integrated sensors. These sensors can detect fluctuations in thyroid hormone levels, metabolic activity, and other relevant biomarkers. The carbon-coded material is programmed to respond to these signals, adjusting the pump's operation accordingly.

In certain embodiments, the device is made of titanium. The device is approximately 5 mm by 20 mm. The device contains a thyroxine store supply of 20,000 mcg of thyroxine. The device contains a microprocessor with embedded signal software. The signal software triggers a pump within the device to inject 100 mcg of thyroxine to the body every 12 hours.

In embodiments, the present disclosure provides methods and a device for thyroxine delivery. This device is designed for subcutaneous implantation to ensure continuous delivery of thyroxine. It features a reservoir regulated by an embedded microprocessor with advanced signal software. The software triggers a precision pump to release a specific dose of thyroxine at regular intervals. This system enhances patient compliance and maintains optimal metabolic control.

In embodiments, the present disclosure provides methods and a device for thyroxine delivery. This device is designed for subcutaneous implantation to ensure continuous, precise delivery of thyroxine. Constructed from biocompatible titanium, it offers durability and resistance to corrosion, ensuring long-term functionality within the body. The device features an internal reservoir that securely stores thyroxine, maintaining its stability and potency over extended periods. Central to its operation is an embedded microprocessor equipped with advanced signal software, which monitors the device's status and the body's hormone levels through integrated sensors. The microprocessor's software is programmed with sophisticated algorithms to control a miniaturized precision pump, which releases specific doses of thyroxine at regular intervals, mimicking the body's natural hormone release patterns. This timed delivery system enhances patient compliance by providing consistent hormone levels, thereby maintaining optimal metabolic control. The device is powered by a rechargeable battery, which can be replenished via transdermal wireless charging, minimizing the need for invasive maintenance. Safety features, including multiple fail-safes and alerts, ensure reliable operation and notify users or healthcare providers of any malfunctions or low thyroxine levels in the reservoir, making this an efficient and patient-friendly solution for thyroxine delivery.

In certain embodiments, the device is constructed from biocompatible titanium, known for its strength and resistance to corrosion. The device dimensions are approximately 5 mm in width and 20 mm in length, making it small and discreet enough for implantation under the skin. At the core of the device is a reservoir that securely stores 20,000 micrograms (mcg) of thyroxine, a crucial hormone for regulating metabolism. The reservoir is designed to maintain the stability and potency of thyroxine over an extended period, ensuring consistent therapeutic effects. Embedded within the device is a sophisticated microprocessor integrated with advanced signal software. This microprocessor continuously monitors the device's operational status and the body's thyroxine levels through a sensor interface. The signal software is programmed with precise algorithms to ensure accurate dosing. The device includes a miniaturized, precision-controlled pump mechanism. The pump is connected to the thyroxine reservoir and can deliver exact doses of the hormone. Every 12 hours, the microprocessor triggers the pump to inject 100 mcg of thyroxine into the body. The injection process is carefully controlled to mimic the body's natural hormone release patterns, minimizing any potential side effects or fluctuations in hormone levels. Additionally, the device features a power-efficient battery system that ensures long-term functionality. The battery is rechargeable through transdermal wireless charging, allowing for maintenance without the need for invasive procedures. Safety features include multiple fail-safes and alerts to notify the user or healthcare provider of any malfunctions or low thyroxine levels in the reservoir. Overall, this titanium device provides a reliable, automated solution for the sustained delivery of thyroxine, enhancing patient compliance and maintaining optimal metabolic control.

In certain embodiments, the device is constructed from biocompatible titanium, known for its strength and resistance to corrosion. Measuring approximately 5 mm in width and 20 mm in length, the device is compact and suitable for subcutaneous implantation. It houses a reservoir containing 20,000 micrograms (mcg) of thyroxine, designed to maintain the hormone's stability and potency over an extended period. At the core of the device is a sophisticated microprocessor embedded with advanced signal software. This microprocessor monitors the device's operational status, and through a sensor interface, the body's thyroxine levels. The software is programmed with precise algorithms to ensure accurate dosing. The device includes a miniaturized, precision-controlled pump mechanism connected to the thyroxine reservoir. Every 12 hours, the microprocessor triggers the pump to inject 100 mcg of thyroxine into the body, closely mimicking the body's natural hormone release patterns. Powering this system is a rechargeable battery, which can be replenished through transdermal wireless charging, eliminating the need for invasive maintenance. Safety features include multiple fail-safes and alerts to notify the user or healthcare provider of any malfunctions or low thyroxine levels in the reservoir. Overall, this titanium device offers a reliable, automated solution for sustained thyroxine delivery, enhancing patient compliance and maintaining optimal metabolic control.

By varying the electrical input to the carbon-coded material, the microprocessor can alter the rate at which the pump dispenses thyroxine. For instance, if the patient's thyroid hormone levels are detected to be lower than the desired threshold, the microprocessor increases the electrical signal to the carbon-coded material, which in turn activates the pump to release a higher dose of thyroxine. Conversely, if hormone levels are within the optimal range, the microprocessor can reduce the signal, slowing down the release rate to maintain steady-state conditions. Additionally, the carbon-coded material can be programmed with specific dosage schedules tailored to the patient's needs. This customization allows for periodic adjustments without the need for invasive procedures. Healthcare providers can remotely update the microprocessor's programming, ensuring that the thyroxine release is continuously optimized for the patient's evolving medical condition. Overall, the integration of carbon-coded material with the microprocessor-controlled pump enables a highly responsive and adaptable hormone delivery system. This approach not only addresses the burst release problem but also provides a scalable solution for personalized medicine, enhancing both the safety and effectiveness of hypothyroidism treatment.

In certain embodiments, the pump for thyroxine in a drug delivery device operates as a critical component ensuring precise and controlled hormone administration. Central to this mechanism is a reservoir that securely stores a concentrated solution of thyroxine, crafted from biocompatible materials to prevent degradation and maintain potency. An embedded microprocessor acts as the control center, utilizing sensors to monitor thyroxine levels in the body, battery status, and the integrity of the pump mechanism. Sophisticated signal software within the microprocessor employs algorithms to regulate the timing and dosage of thyroxine release, mimicking the body's natural hormone release patterns. Upon receiving signals from the software, the precision pump—a miniaturized component capable of delivering exact doses—injects thyroxine at regular intervals. The system also incorporates multiple fail-safes and alert mechanisms to notify users or healthcare providers of any malfunctions or low hormone levels, ensuring reliable and consistent hormone delivery for optimal metabolic control.

In certain embodiments, the present disclosure is a method for thyroid hormone delivery. The method comprises signaling the delivery of the thyroid hormone on a timed basis, wherein the time basis is on a twelve-hour interval. Further, the method includes sending a command to a thyroid hormone pump, loading the thyroid hormone supply by the pump to a secretion chamber, and pumping thyroid hormones from a secretion chamber 403 to the human body by the thyroid hormone pump.

In certain embodiments, the present disclosure is an apparatus for thyroid hormone delivery. In such embodiments, the apparatus is an implantable device with a cylindrical shape 100. The implantable device further comprises a microprocessor 100 with embedded instructions for commanding a thyroid hormone pump. Further the apparatus includes a thyroid hormone pump, receiving a signal from the microprocessor to pump thyroid hormone supply and a thyroid hormone supply. Finally, the thyroid hormone supply is pumped into the human body by a thyroid hormone pump 303.

In certain embodiments, the present disclosure is a method for thyroid hormone delivery. The method comprises signaling the delivery of the thyroid hormone on a timed basis 300. The method further comprises sending a command to a thyroid hormone pump 201, delivering thyroid hormones to the human body, and optimizing thyroid hormone homeostasis in the human body 204.

It is to be understood that while certain embodiments and examples of the invention are illustrated herein, the invention is not limited to the specific embodiments or forms described and set forth herein. It will be apparent to those skilled in the art that various changes and substitutions may be made without departing from the scope or spirit of the invention and the invention is not considered to be limited to what is shown and described in the specification and the embodiments and examples that are set forth therein. Moreover, several details describing structures and processes that are well-known to those skilled in the art and often associated with biotechnologies are not set forth in the following description to better focus on the various embodiments and novel features of the disclosure of the present invention. One skilled in the art would readily appreciate that such structures and processes are at least inherently in the invention and in the specific embodiments and examples set forth herein.

One skilled in the art will readily appreciate that the present invention is well adapted to carry out the objectives and obtain the ends and advantages mentioned herein as well as those that are inherent in the invention and in the specific embodiments and examples set forth herein. The embodiments, examples, methods, and compositions described or set forth herein are representative of certain preferred embodiments and are intended to be exemplary and not limitations on the scope of the invention. Those skilled in the art will understand that changes to the embodiments, examples, methods and uses set forth herein may be made that will still be encompassed within the scope and spirit of the invention. Indeed, various embodiments and modifications of the described compositions and methods herein which are obvious to those skilled in the art, are intended to be within the scope of the invention disclosed herein. Moreover, although the embodiments of the present invention are described in reference to use in connection with biotechnology, ones of ordinary skill in the art will understand that the principles of the present inventions could be applied to other types of biotechnology for a wide variety of applications.

1. A method for thyroid hormone delivery, the method comprising:
signaling the delivery of the thyroid hormone on a timed basis, wherein the time basis is on a twelve-hour interval,
sending a command to a thyroid hormone pump,
loading the thyroid hormone supply by the pump to a secretion chamber,
pumping thyroid hormones from a secretion chamber to the human body by the thyroid hormone pump, and
optimizing metabolic homeostasis in the human body.

2. The method of claim 1, wherein the thyroid hormones delivered to the human body are a continuous supply of thyroxine.

3. The method of claim 1, wherein the thyroid hormones delivered to the human body are a continuous supply of thyroxine and triiodothyronine, wherein the ration of the continuous supply is thirteen parts thyroxine to one part triiodothyronine.

4. The method of claim 1, wherein the time basis is on a twenty-four-hour interval.

5. The method of claim 1, wherein metabolic homeostasis is optimized by ensuring a balanced and continuous delivery of thyroxine to the human body, stabilizing the concentration of thyroid hormone for the human body.

6. An apparatus for thyroid hormone delivery, the apparatus comprising:
an implantable device with a cylindrical shape,
the implantable device further comprising:
a microprocessor with embedded instructions for commanding a thyroid hormone pump,
a thyroid hormone pump, receiving a signal from the microprocessor to pump thyroid hormone supply,
a thyroid hormone supply,
the thyroid hormone supply being pumped into the human body by the thyroid hormone pump.

7. The apparatus of claim 6, wherein the thyroid hormones being pumped to the human body are a continuous supply of thyroxine.

8. The apparatus of claim 6, wherein the thyroid hormones being pumped to the human body are a continuous supply of thyroxine and triiodothyronine, wherein the ration of the continuous supply is thirteen parts thyroxine to one part triiodothyronine.

9. The apparatus of claim 6, wherein the thyroid hormone supply is 20,000 micrograms of thyroxine.

10. The apparatus of claim 6, wherein the implantable device is a cylinder with measurements of approximately 5 mm by 20 mm.

11. The apparatus of claim 6, wherein the implantable device is a cylinder with measurements of approximately 5 mm by 20 mm.

12. The apparatus of claim 6, wherein the implantable device is a cylinder with measurements of approximately 7 mm by 24 mm.

13. The apparatus of claim 6, wherein the implantable device is a cylinder with measurements of approximately 3 mm by 15 mm.

14. The apparatus of claim 6, wherein the implantable device is a non-cylinder with measurements of approximately 3 mm by 15 mm.

15. A method for thyroid hormone delivery, the method comprising:
signaling the delivery of the thyroid hormone on a timed basis,
sending a command to a thyroid hormone pump,
pumping the thyroid hormone by a thyroid hormone pump,
delivering thyroid hormones to the human body, and
maintaining thyroid hormone homeostasis in the human body.

16. The method of claim 15, wherein the thyroid hormones delivered to the human body are a continuous supply of thyroxine.

17. The method of claim 15, wherein the thyroid hormones delivered to the human body are a continuous supply of thyroxine and triiodothyronine, wherein the ration of the continuous supply is thirteen parts thyroxine to one part triiodothyronine.

18. The method of claim 15, wherein the method is used for delivery of any non-thyroid hormone.

19. The method of claim 15, wherein the method is used for delivery of any thyroid hormone.

20. The method of claim 15, wherein the method is used for delivery of any combination of thyroid and non-thyroid hormone.
